(19) United States
(12) Patent Application Publication
(10) Pub. No.: US 2023/0148041 A1
(43) Pub. Date: May 11, 2023

(54) TRANSISTOR TYPE SENSOR

(71) Applicants: THE UNIVERSITY OF TOKYO, Tokyo (JP); JNC CORPORATION, Tokyo (JP)

(72) Inventors: Tsuyoshi MINAMI, Tokyo (JP); Qi ZHOU, Tokyo (JP); Yusuke YAMANASHI, Chiba (JP); Katsumasa NAKAHARA, Chiba (JP); Hirokazu NAGAOKA, Chiba (JP)

(73) Assignees: THE UNIVERSITY OF TOKYO, Tokyo (JP); JNC CORPORATION, Tokyo (JP)

(21) Appl. No.: 17/965,759

(22) Filed: Oct. 13, 2022

(30) Foreign Application Priority Data
Oct. 20, 2021 (JP) 2021-171511

(51) Int. Cl. G01N 27/414

(52) U.S. Cl. CPC G01N 27/4145; G01N 27/4148; G01N 33/74

(57) ABSTRACT

A small transistor type sensor capable of detecting a specific compound such as oxytocin is provided. The transistor type sensor includes a detection electrode that detects a compound by capturing the compound, and a field effect transistor that has a gate electrode connected to the detection electrode, wherein a surface of the detection electrode is provided with a film of a molecularly imprinted polymer having a space to which the compound is allowed to bond.

### CROSS-REFERENCE TO RELATED APPLICATION

This application claims the priority benefit of Japanese Application Serial No. 2021-171511, filed on Oct. 20, 2021. The entirety of each of the above-mentioned patent applications is hereby incorporated by reference herein and made a part of this specification.

### BACKGROUND

### Technical Field

The disclosure relates to a field effect transistor type sensor that includes a detection electrode having a molecularly imprinted polymer, a method for manufacturing a field effect transistor type sensor, and a method for quantitatively measuring a compound using a field effect transistor type sensor.

### Description of Related Art

Oxytocin is a hormone substance secreted in the brain and can be used as a biomarker to objectively evaluate the mental state of a mother in maternity nursing.

Enzyme-linked immunosorbent assay (ELISA) and high-performance liquid chromatography (HPLC) are used as techniques for analyzing oxytocin, but they require highly specialized knowledge, sample pretreatment, and large-scale equipment. Furthermore, since the long measurement time is also a factor that hinders rapid inspection, there is a demand to embody a small sensor that easily and rapidly achieves high-sensitivity detection. In addition, since a change in an amount of the oxytocin is evaluated, quantitative detection is required.

Further, in the related art, as a small sensor, a sensor using a transistor as in Non-Patent Document 1 has been examined, but a method for measuring oxytocin using such a small sensor cannot be realized.

### Non-Patent Documents

[Non-Patent Document 1] Minami, T. ACS Sens. 2019, 4, 2571-2587

The present inventors have examined a detection method using a small transistor type sensor as a method for detecting oxytocin. As a result, the present inventors have discovered that a sensor which uses a field effect transistor including a detection electrode connected to a gate electrode and includes a molecularly imprinted polymer on a surface of the detection electrode can quantitatively detect a compound and have thus arrived at the disclosure.

### SUMMARY

That is, an embodiment of the disclosure includes the following.

A transistor type sensor including:
a detection electrode that detects a compound by capturing the compound; and
a field effect transistor that has a gate electrode connected to the detection electrode,
wherein a surface of the detection electrode is provided with a film of a molecularly imprinted polymer having a space to which the compound is allowed to bond.

The transistor type sensor according to [1], wherein each space is formed such that each compound is allowed to be captured in the same orientation.

The transistor type sensor according to [ 1 ] or [2], wherein a non-covalently bondable functional group of the molecularly imprinted polymer is exposed in the space toward the captured compound.

The transistor type sensor according to [1], wherein the molecularly imprinted polymer film is formed of a monomer having an amino group-derived moiety.

The transistor type sensor according to [1], wherein the compound includes a —SH group, a —S—S— bond, or a —C═C—H group.

The transistor type sensor according to [5], wherein the compound is oxytocin.

The transistor type sensor according to [1], wherein the field effect transistor is an organic semiconductor transistor.

The transistor type sensor according to [1], wherein the field effect transistor is a p-type semiconductor.

The transistor type sensor according to [1], wherein the transistor type sensor detects the detection target in a solution or dispersion liquid.

A manufacturing method for manufacturing the field effect transistor type sensor according to [1],
wherein the detection electrode is obtained by a method including:
a step of chemically bonding the compound to a surface of a main body of the detection electrode;
a step of applying a monomer-containing solution to the main body of the detection electrode or immersing the main body of the detection electrode in the monomer-containing solution to form the molecularly imprinted polymer;
a step of polymerizing each monomer in the presence of the main body of the detection electrode to form a polymer; and
a step of removing the compound covered with the polymer and chemically bonded to the surface of the main body of the detection electrode to form the molecularly imprinted polymer.

The manufacturing method according to [10], wherein the polymerization is electrolytic polymerization.

The manufacturing method according to [10], wherein the removal of the compound covered with the molecularly imprinted polymer and chemically bonded to the surface of the main body of the detection electrode is performed by an electrochemical reaction.

A measurement method for measuring a compound to be detected using the field effect transistor type sensor according to [1],
wherein the transistor type sensor further includes a counter electrode, and
wherein the detection electrode and the counter electrode are brought into contact with the detection target.

The measurement method according to [13], wherein detection is performed in a state where the compound to be detected is included in a solution or a dispersion liquid and the detection electrode and the counter electrode are put into the solution or the dispersion liquid.

The measurement method according to [13], wherein an applied voltage is a DC voltage.

A measurement method for quantitatively measuring a compound using a field effect transistor type sensor, including:
a step of bringing a compound to be detected into contact with the detection electrode of the field effect transistor type sensor according to [1];
a step of measuring a current Id flowing between a drain electrode and a source electrode of the transistor for each concentration of each compound to obtain a concentration-current Id relationship curve;
a step of bringing the compound having an unknown concentration into contact with the detection electrode to obtain a current Id of the compound having an unknown concentration; and
a step of comparing the current Id of the compound having an unknown concentration with the concentration-current Id relationship curve to determine a concentration for the compound from the current Id having an unknown concentration.

A measurement method for quantitatively measuring a compound using a field effect transistor type sensor, including:
a step of bringing a compound to be detected into contact with the detection electrode of the field effect transistor type sensor according to [1];
a step of measuring a threshold voltage for each concentration of each compound to obtain a concentration-threshold voltage relationship curve;
a step of bringing the compound having an unknown concentration into contact with the detection electrode to obtain a threshold voltage of the compound having an unknown concentration; and
a step of comparing the threshold voltage of the compound having an unknown concentration with the concentration-threshold voltage relationship curve to determine a concentration for the compound from the threshold voltage having an unknown concentration.

The measurement method according to [16],
wherein the measuring of the threshold voltage includes
a step of applying a voltage Vd to a drain electrode with a source electrode of the field effect transistor as a reference and applying a voltage Vg to a counter electrode with the source electrode as a reference;
a step of sweeping the Vg and measuring a current Id flowing between the drain electrode and the source electrode to obtain a Vg-Id curve; and
a step of using the Vg-Id curve to obtain a value of the threshold voltage.

The measurement method according to [16] or [17],
wherein the transistor type sensor further includes a counter electrode, and
wherein the detection electrode and the counter electrode are brought into contact with the compound.

The measurement method according to [16] or [17], wherein detection is performed in a state where the compound is included in a solution or a dispersion liquid and the detection electrode and the counter electrode are put into the solution or the dispersion liquid.

The measurement method according to [16] or [17], wherein an applied voltage is a DC voltage.

### BRIEF DESCRIPTION OF THE DRAWINGS

FIG. 1 shows a schematic diagram of a transistor type sensor of Example 1.

FIG. 2 is a schematic diagram showing the formation of a molecularly imprinted polymer on an electrode surface.

FIG. 3 is a manufacturing procedure of the transistor type sensor of Example 1.

FIG. 4 is a diagram showing measurement results of Example 1.

FIG. 5 is a diagram showing measurement results of Example 1.

FIG. 6 is a diagram showing a change in a threshold voltage with respect to a concentration in Example 1.

FIG. 7 is a diagram in which a current Id is plotted for each oxytocin concentration in a detection experiment of Example 1.

FIG. 8 is a diagram showing measurement results of Comparative Example 1.

FIG. 9 is a diagram showing measurement results of Example 2.

FIG. 10 is a diagram showing measurement results of Example 3.

### DESCRIPTION OF THE EMBODIMENTS

Since the transistor type sensor including the detection electrode having the molecularly imprinted polymer of the disclosure has the molecularly imprinted polymer on the surface of the detection electrode, it is possible to selectively detect the compound to be detected by providing a space corresponding to the compound to be detected in the molecularly imprinted polymer.

Further, since the molecularly imprinted polymer is formed such that the compound to be detected is allowed to be captured in the same orientation, it is possible to more selectively detect the compound to be detected.

Moreover, even in a case where there is a plurality of types of compounds to be detected, it is possible to quantitatively measure each compound by providing a space corresponding to the detection target in the molecularly imprinted polymer.

Furthermore, since detection can be performed when the molecularly imprinted polymer is provided on the detection electrode, manufacture is easy, and the measurement method is also simple.

### Regarding Transistor Type Sensor

An organic semiconductor transistor type sensor of the disclosure includes a detection electrode that detects a compound by capturing the compound, and a field effect transistor that has a gate electrode connected to the detection electrode, wherein a surface of the detection electrode is provided with a film of a molecularly imprinted polymer having a space to which the compound is allowed to bond. The detection electrode is an extended gate electrode of the transistor.

### Transistor

A sensor of the disclosure includes a field effect transistor. A thin film transistor made of an organic semiconductor is preferable because it is small and can be used easily.

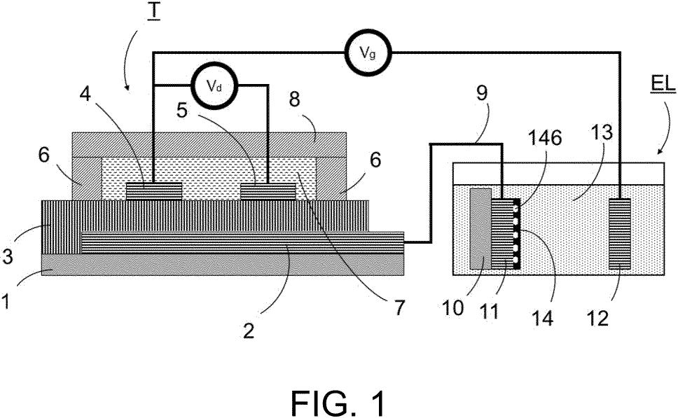

In the disclosure, a field effect transistor having a normal configuration can be used, an example of which is shown in FIG. 1. A field effect transistor T in FIG. 1 is a typical field effect transistor and is constituted by a substrate 1, a gate electrode 2, a gate insulation film 3, a source electrode 4, a drain electrode 5, a bank 6, a semiconductor thin film 7, and a sealing film 8.

Materials forming the field effect transistor T are not particularly limited either. For example, the substrate 1 may be made of inorganic materials such as glass, ceramics, and a metal as well as organic materials such as a resin and paper. As the gate electrode 2, aluminum, silver, gold, copper, titanium, indium tin oxide (ITO), poly(3,4-ethylenedioxythiophene), polystyrene sulfonate, conductive carbon nanotubes, graphene, a conductive organic-inorganic composite material, or the like may be used. Examples of a material forming the gate insulation film 3 include silica (silicon oxide), alumina (aluminum oxide), a self-assembled monomolecular film, polystyrene, polyvinyl phenol, polyvinyl alcohol, polymethyl methacrylate, polydimethylsiloxane polysilsesquioxane, an ionic liquid, polytetrafluoroethylene, and the like. The substrate 1 and the gate electrode 2 may be integrated with each other, and a metal substrate or Si substrate may be used. The Si substrate is preferably doped in order to improve its conductivity. In a case where the semiconductor thin film is a p-type, a substrate doped in an n-type may be used, and in a case where the semiconductor thin film is an n-type, a substrate doped in a p-type may be used. Furthermore, as the gate insulation film 3, SiOz formed by oxidizing a surface of the Si substrate may be used. Examples of a material of each of the source electrode 4 and the drain electrode 5 include metals such as gold, silver, copper, platinum, and aluminum, conductive polymers such as PEDOT:PSS, conductive carbon nanotubes, graphene, conductive organic-inorganic composite materials, and the like. Examples of a material forming the bank 6 include polytetrafluoroethylene, and examples of a material forming the sealing film 8 include polytetrafluoroethylene, polyparaxylylene, and the like.

The substrate 1, the gate electrode 2, the gate insulation film 3, the source electrode 4, and the drain electrode 5 may be surface-treated, for example, a self-assembled monomolecular film may be formed to adjust liquid repellency of a surface.

A material of the semiconductor thin film 7 is not particularly limited as long as the material can exhibit a function of the semiconductor thin film, but in a case where the semiconductor thin film 7 is an organic semiconductor and a P-type, pentacene, dinaphthothienothiophene, benzothienobenzothiophene (Cn-BTBT), and TIPS pentacene, TES-ADT, rubrene, P3HT, PBTTT, or the like may be used, and in the case where the semiconductor thin film 7 is an organic semiconductor and a N-type, fullerene or the like may be used. Among them, the following compound or the like is preferably used and is also used as a semiconductor material in examples described in this specification.

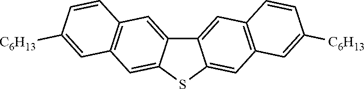

In FIG. 1, a detection electrode EL includes a conducting wire 9, a detection electrode substrate 10, a detection electrode main body 11, a counter electrode 12, an aqueous solution 13 containing a detection target, and a film 14 of a molecularly imprinted polymer having a space 146 corresponding to a compound to be detected. The detection electrode main body 11 is electrically connected to the gate electrode 2 of the transistor T via the conducting wire 9. Experimentally, it is preferable to put the detection electrode EL and the counter electrode into a tube including the aqueous solution in order to facilitate detection of a liquid.

Examples of a material of the detection electrode substrate 10 include polyethylene naphthalate and the like. The detection electrode main body (an extended gate electrode main body) 11 is disposed on a surface of the detection electrode substrate 10. However, in a case where the detection electrode main body 11 is self-supporting, the detection electrode main body 11 may also serve as the detection electrode substrate 10. Similar to the gate electrode 2, as a material of the detection electrode main body 11, aluminum, silver, gold, copper, titanium, indium tin oxide (ITO), poly(3,4-ethylenedioxythiophene), polystyrene sulfonate, conductive carbon nanotubes, graphene, a conductive organic-inorganic composite material, or the like may be used. In a case where a polymer is formed by electrolytic polymerization, the material preferably has high chemical stability, and gold, conductive carbon nanotubes, or graphene is preferably used.

On a surface of the detection electrode main body 11, a metal thin film having a thickness of 10 nm to 1000 µm and formed of gold or the like, or a thin film of carbon nanotubes, graphene, a conductive inorganic material, or a conductive organic material is preferably formed, and a metal oxide film having a thickness of 1 nm to 1000 nm, preferably 1 nm to 50 nm and formed of SiO₂ or the like may be formed. In this specification, the surface of the detection electrode main body 11 is a concept that includes both a surface which is the material of the detection electrode main body 11 itself and a surface on which the metal thin film or the metal oxide film is formed.

The counter electrode 12 only has to be formed of a conductive material, and a metal electrode or a carbon electrode may be used as the counter electrode 12. In the counter electrode 12, a conductive film may be formed on a substrate formed of a material equivalent to that of the detection electrode substrate 10, or a conductive film may be formed on a detection electrode substrate in common with the detection electrode main body. Further, a film of a material that is the same as or different from that of the film 14 of a molecularly imprinted polymer may be formed on the counter electrode 12. The film on the counter electrode 12 may be subjected to the same treatment as the film 14 of a molecularly imprinted polymer, or may be subjected to a part of the treatment of the film 14 of a molecularly imprinted polymer. Moreover, a reference electrode may be used, and examples of the reference electrode include an electrode made of Ag/AgCl or the like which can be generally used.

The detection electrode main body 11 includes the film 14 of a molecularly imprinted polymer having the space 146 corresponding to the compound to be detected on the surface of the detection electrode main body 11. The compound to be detected enters this space 146 and interacts with the detection electrode.

The film 14 of a molecularly imprinted polymer provided on the surface of the detection electrode main body 11 will be described.

A type of a polymer of the film 14 of a molecularly imprinted polymer is not particularly limited, but the polymer preferably has a non-covalently bondable functional group capable of interacting with the compound to be detected in a non-covalent bond inside. That is, the film 14 of a molecularly imprinted polymer preferably has a non-covalently bondable functional group in a main chain or branched chain of the polymer. Examples of the non-covalently bondable functional group include, but are not particularly limited to, an OH group and the like.

A monomer, which is a material for forming the film 14 of a molecularly imprinted polymer, preferably has a double bond for polymerization. The double bond may be present in a molecular chain or may be a double bond in an aromatic ring.

Furthermore, a monomer having an amino group is preferable from the viewpoint that an appropriate polymerization reaction can be performed.

Therefore, the monomer for forming the film 14 of a molecularly imprinted polymer is preferably a compound having a double bond, an OH group, and an amino group.

As the monomer for forming the film 14 of a molecularly imprinted polymer, one type of monomers may be used, or two or more types of monomers may be used, and it is preferable that different monomers form a polymer. In addition, in a case where two or more types of monomers are used, not all the monomers need to have a non-covalently bondable functional group.

Examples of the monomer for forming the film 14 of a molecularly imprinted polymer include ortho-phenylenediamine, acrylamide, N,N′-methylenebisacrylamide, aniline, an aminophenylboronic acid, pyrrole, aminophenol, an aminobenzoic acid, dopamine, and the like.

Among them, dopamine is preferable because it facilitates formation of a suitable film 14 of a molecularly imprinted polymer.

The monomer for forming the film 14 of a molecularly imprinted polymer is not limited to be water-soluble or water-insoluble, but from the viewpoint of ease of handling, the monomer is preferably a water-soluble monomer.

The space 146 of the film 14 of a molecularly imprinted polymer is a space corresponding to a shape of the compound to be detected. Although it is preferable to use one type of a compound to be detected in terms of accuracy of detection, it is theoretically possible to detect two or more types of compounds. In that case, a plurality of types of spaces corresponding to the compounds to be detected is created in film 14 of a molecularly imprinted polymer.

The compound to be detected is not particularly limited as long as it is a compound that can chemically bond to the surface of the detection electrode, and a compound including a -SH group, a —S—S— bond, or a —C═C—H group is preferable in that a chemical bond can be formed on the detection electrode.

The compound including a —SH group, a —S—S— bond, or a —C═C—H group is preferably oxytocin.

Although there may be a plurality of functional groups that chemically bonds to the detection electrode, the compounds to be detected are preferably aligned in the same orientation and preferably have a structure to specifically bond to the detection electrode.

In a case where the compound to be detected does not include a —SH group, a —S—S—bond, or a —C═C—H group, a method in which a linking compound including a —SH group, a —S—S— bond, or a —C═C—H and for linking the compound to be detected and the electrode main body first chemically bonds to the detection electrode, and then the compound to be detected bonds to the linking compound on the surface of electrode is enumerated. Alternatively, the linking compound and the compound to be detected may bond to each other in advance, and then the bonded compound and compound to be detected may bond to the electrode main body.

The transistor type sensor of the disclosure performs measurement when the detection electrode is immersed in an aqueous solution of the compound to be detected, for example. A value of a threshold voltage changes depending on a concentration of the compound to be detected. Therefore, after a relational curve between the threshold voltage and the concentration in advance is created, a threshold voltage of an aqueous solution having an unknown concentration is measured, and the concentration is calculated from that value.

In addition to the value of the threshold voltage value, a current value can also be used for measurement. The current value can be calculated by simply comparing a measured Id with a predetermined voltage Vg. However, the amount of a change in Id is often not linear with respect to the voltage Vg, and the accuracy of measurement may vary depending on which voltage Vg Id is used for calculation. Further, instead of an absolute voltage being set arbitrarily, in a case where current values of transistors having different threshold voltages are compared with each other, the maximum value of the current, a median value of the current, a current value at a voltage (Vth + Vd), and the like may be set.

### Regarding Method for Manufacturing Transistor Type Sensor

### Manufacture of Detection Electrode

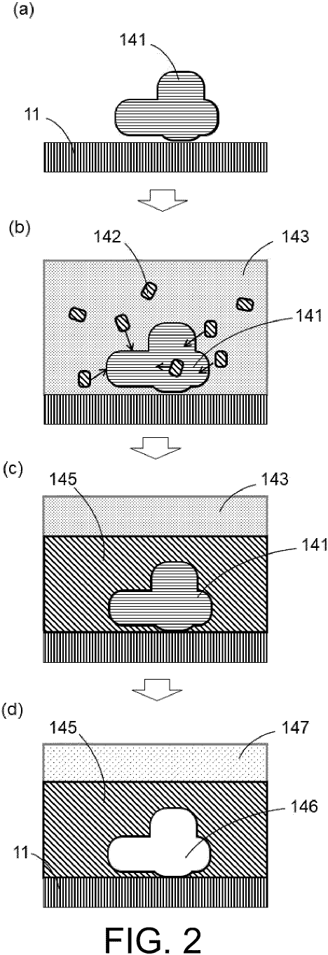

Manufacture of the detection electrode provided with the film 14 of a molecularly imprinted polymer will be described with reference to FIG. 2.

The film 14 of a molecularly imprinted polymer is obtained by a method including a step of chemically bonding a compound 141 to be detected to a surface of a main body 11 of the detection electrode ((a) of FIG. 2), a step of applying a monomer-containing solution 143 that contains a monomer 142 to the main body 11 of the detection electrode or immersing the main body 11 of the detection electrode in the monomer-containing solution 143 to form the molecularly imprinted polymer ((b) of FIG. 2), a step of polymerizing each monomer 142 in the presence of the main body 11 of the detection electrode to form a polymer 145 ((c) of FIG. 2), and a step of removing the compound 141 covered with the polymer 145 and chemically bonded to the surface of the main body 11 of the detection electrode to form the molecularly imprinted polymer ((d) of FIG. 2).

First, the compound 141 to be detected is chemically bonded onto the detection electrode ((a) of FIG. 2). By bringing the surface of the main body 11 of the detection electrode into contact with the solution containing the compound 141 to be detected, it is possible to chemically bond the compound 141 to be detected to the surface of the main body 11 of the detection electrode. In general, the detection electrode is immersed in the solution.

In a case where the compound to be detected does not have the functional group that chemically bonds to the detection electrode, the surface of the main body 11 of the detection electrode may be modified with a molecule for bonding to the compound 141 to be detected. This molecule preferably has two functional groups, one of which bonds to the surface of the detection electrode, and the other of which bonds to the compound 141 to be detected.

Further, in order to align the orientations of each compound 141 to be detected, a molecule that does not bond to the compound 141 to be detected may be disposed on the surface of the main body 11 of the detection electrode prior to the compound 141 to be detected.

In this way, when the compound 141 to be detected is chemically bonded to the surface of the main body 11 of the detection electrode, the compounds 141 to be detected are disposed in the same orientation, thereby it is possible to perform measurement with excellent sensitivity in actual detection measurement.

Next, the monomer-containing solution 143 that contains the monomer 142 is applied to the main body 11 of the detection electrode or the main body 11 of the detection electrode is immersed in the monomer-containing solution to form the film 14 of the molecularly imprinted polymer. That is, the monomer-containing solution 143 is brought into contact with the main body 11 of the detection electrode in a state where the compound 141 is chemically bonded onto the detection electrode ((b) of FIG. 2). As a contact method, an applying method or an immersion method mentioned here is preferable in that the monomer solution can be brought into contact with the main body of the detection electrode uniformly.

The monomer-containing solution 143 is preferably an aqueous solution from the viewpoint of ease of handling and environmental impact. The concentration of the monomer in the aqueous solution is preferably 10 to 5000 µM, more preferably 50 to 1000 µM, and even more preferably 100 to 500 µM.

Although a temperature during the immersion is not particularly limited, the immersion is preferably performed at a normal temperature.

Next, the monomers are polymerized in the presence of the detection electrode main body 11 to form the polymer 145 ((c) of FIG. 2). The polymer 145 is formed around the compound 141 by polymerizing the monomers in the presence of the main body 11 of the detection electrode and by polymerizing the monomers in a state where the compound to be detected is chemically bonded onto the main body 11 of the detection electrode.

At this time, it is preferable that the monomers are polymerized such that a main chain or branched chain of the polymer 145 has a functional group with respect to the compound 141 to be detected. By inducing the functional group that interacts with the compound 141 to be detected in the main chain or branched chain of the polymer 145, it easier for the compound 141 to be detected to enter the pores (the space 146) in the intended orientation.

The polymerization is preferable in that the polymer can be formed uniformly to form a film on the detection electrode through electrolytic polymerization. In the electrolytic polymerization, a technique of cyclic voltammetry is used, a working electrode and a counter electrode are scanned with a potential applied therebetween with a potential of a reference electrode as a reference, the potential reaches a potential for a chemical reaction, and a polymer is formed. By repeating this several times, a polymer is formed, and a film is grown. In the technique of cyclic voltammetry, the working electrode is the detection electrode, the counter electrode is preferably made of platinum, and the reference electrode is preferably made of Ag/AgCl.

The potential applied between the working electrode and the counter electrode during the polymer formation is preferably -0.5 to 1.5 V, more preferably -0.5 to 1.0 V, and even more preferably -0.5 to 0.5 V.

Finally, the compound 141 to be detected which is covered with the polymer 145 and chemically bonded to the surface of the main body 11 of the detection electrode is removed to form the molecularly imprinted polymer. By removing the compound 141 to be detected, it is possible to form the space 146 corresponding to the compound such that the space 146 in the shape of the compound is arranged to line up with the surface of the main body 11 of the detection electrode and the functional group of the molecularly imprinted polymer is exposed in the space 146. By using the main body 11 of the detection electrode, it is possible to detect the compound to be detected with high sensitivity.

The removal includes a method of immersion in a basic solution to break the chemical bond between the compound 141 to be detected and the surface of the main body 11 of the detection electrode. Examples of the basic solution include an aqueous solution of potassium hydroxide and an aqueous solution of sodium hydroxide, and the pH is preferably 9 or more, more preferably 10 to 15, and even more preferably 11 to 13.

Although a temperature during the immersion is not particularly limited, the immersion is preferably performed at a normal temperature.

In order to sufficiently remove the compound 141 to be detected, it is preferable to remove it using an electrochemical method. In the removal performed by the electrochemical method, a technique of cyclic voltammetry is used, a working electrode and a counter electrode are scanned with a potential applied therebetween with a potential of a reference electrode as a reference, the potential reaches a potential to break the chemical bond, and the removal is performed. By repeating this several times, it is possible to perform the removal sufficiently or completely. The process of removing the compound 141 can be checked by monitoring the potential.

The potential applied between the working electrode and the counter electrode during the removal is preferably +0.5 to -1.5 V, more preferably 0 to 1.3 V, and even more preferably -0.3 to 1.0 V.

Using the detection electrode obtained in this way, the transistor type sensor as described below is manufactured.

### Manufacture of Organic Semiconductor Transistor

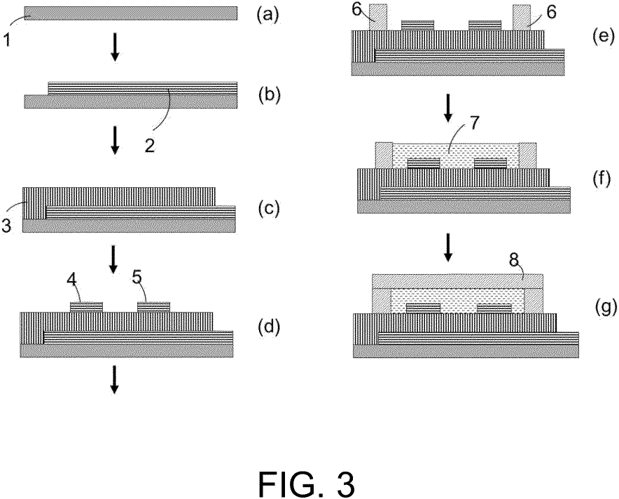

An example of a method for manufacturing the field effect transistor T shown in FIG. 1 will be described with reference to FIG. 3. First, the substrate 1 (made of glass) is prepared ((a) of FIG. 3), and the gate electrode 2 (made of aluminum) having a thickness of 30 nm is formed thereon ((b) of FIG. 3). Then, RIE treatment (forming an aluminum oxide film through reactive ion etching treatment) is performed for 15 minutes, and the gate insulation film 3 is formed through treatment with HFPA ((c) of FIG. 3). Further, the source and drain electrodes 4 and 5 (both made of gold) are formed through patterning ((d) of FIG. 3). Thereafter, the bank 6 (made of polytetrafluoroethylene) is formed ((e) of FIG. 3), and a layer of the semiconductor thin film 7 is formed ((f) of FIG. 3). Finally, the sealing film 8 (made of polytetrafluoroethylene) is formed through spin coating or the like ((g) of FIG. 3), and thus the field effect transistor T is manufactured.

The transistor type sensor can be manufactured by connecting the gate electrode 2 and the detection electrode EL to each other.

The present sensor detects a substance to be detected at a concentration of preferably 0.1 to 1000 pg/mL, and more preferably 10 to 200 pg/mL. If the concentration exceeds 1000 pg/mL, the threshold voltage does not shift, and thus qualitative detection is possible, but quantification is difficult. Moreover, if the concentration is less than 0.1 pg/mL, detection limit is exceeded, and thus not only the quantification but also the qualitative detection is difficult.

In this example, a temperature at the time of the detection is not particularly limited, but the detection can be performed at a room temperature. In addition, a pressure at the time of the detection is also not particularly limited, but the detection may be performed in the air. Further, it is preferable that the substance to be detected have the above concentration, and thus it is preferable that the detection be performed by immersing the detection electrode in the detection target-containing solution.

### Detection Method

In a method for measuring the detection target, the Vd of -1.0 V is applied, the Vg from +0.5 V to -3.0 V is applied for each step of 0.1 V, and Id is measured at that time. Then, Id^{½} and Vg draw an approximate straight line in a linear region (a saturation region), and a threshold voltage is obtained by calculating a value of an X-intercept thereof. The obtained threshold voltage changes depending on the concentration of the detection target.

A threshold voltage in the detection target whose concentration is unknown is obtained by drawing a curve of the concentration to the threshold voltage, and thus it is possible to measure the concentration.

### EXAMPLES

The disclosure will be described in more detail on the basis of examples below, but the disclosure is not limited to these examples.

### Electrode Manufacture Example 1

A thin film of gold having a thickness of 100 nm was formed by a vacuum deposition method using a metal mask on a polyethylene naphthalate (PEN) substrate to obtain Electrode 1. Treatment liquid 1 in which oxytocin was dissolved in Dulbecco phosphate-buffered saline such that the concentration became 250 µM was prepared. Next, the electrode 1 was washed with ethanol and ultrapure water, dried, immersed in Treatment liquid 1 at room temperature for 24 hours, washed with ultrapure water, and dried to obtain Electrode 2.

Then, 0.6 mM of dopamine hydrochloride was added to Dulbecco phosphate-buffered saline to prepare Treatment liquid 2. Treatment liquid 2 was put into a container, Electrode 2, a platinum counter electrode, and a Ag/AgCl reference electrode were immersed, and a voltage was applied using cyclic voltammetry. A voltage range was set to -0.5 to 0.5 V, a scan speed was set to 20 mV/s, and this was repeated for 9 cycles. Then, Electrode 2 was washed with ultrapure water and dried to obtain Electrode 3.

Furthermore, potassium hydroxide was added to ultrapure water such that the concentration became 0.01 M to prepare Treatment liquid 3. Treatment liquid 3 was put into a container, Electrode 3, a platinum counter electrode, and a Ag/AgCl reference electrode were immersed, and a voltage was applied using cyclic voltammetry. A voltage range was set to -0.3 to -1.0 V, a scan speed was set to 20 mV/s, and this was repeated for 5 cycles. Then, Electrode 3 was washed with ultrapure water and dried to obtain a polymer-immobilized detection electrode (the detection electrode of the examples) having a space shaped like an oxytocin molecule inside.

### Example 1

### Manufacture of Transistor Type Sensor using Polymer-Immobilized Detection Electrode

The field effect transistor type sensor of the disclosure was manufactured by connecting the polymer-immobilized detection electrode obtained in Electrode manufacture example 1 to the gate electrode of the field effect transistor using the organic semiconductor obtained by the manufacturing method shown in FIG. 3. In the transistor type sensor of this example, a gate terminal (not shown) of a semiconductor parameter analyzer was connected to the counter electrode (Ag/AgCl).

### Check of Operation of Field Effect Transistor

The gate electrode, the source electrode, and the drain electrode of the field effect transistor using the organic semiconductor obtained in Example 1 were connected to a gate terminal, a source terminal, and a drain terminal of the semiconductor parameter analyzer (terminals not shown), respectively.

Electrical measurement was performed with a source-drain voltage (Vd) set to -1.0 V and a gate voltage (Vg) set to 0.5 to -3 V, and thus it was checked that the transistor was driven with the Vg within -3 V, a source-gate current (Ig) was smaller than a source-drain current (Id) by one order or more, and the threshold voltage shift was sufficiently small.

### Oxytocin Detection Experiment: Check of Threshold Voltage

Each manufactured electrode was immersed in an oxytocin solution adjusted to each concentration of 10, 20, 30, 40, 50, 60, 70, 80, 90, and 100 pg/mL in a microtube and allowed to stand for 30 minutes. This operation was set such that an antigen-antibody reaction proceeded sufficiently.

A measurement experiment was performed at each concentration.

In the oxytocin solution included in the microtube, an oxytocin polymer-immobilized detection electrode was connected to the gate electrode of the field effect transistor using the organic semiconductor obtained by the manufacturing method shown in FIG. 3, and the counter electrode (Ag/AgCl) was put thereinto and connected to the gate terminal (not shown) of the semiconductor parameter analyzer.

In the measurement, the source-drain voltage (Vd) was set to -1.0 V, and the gate voltage (Vg) was set to 0.5 to -3 V. The measurements were performed three times.

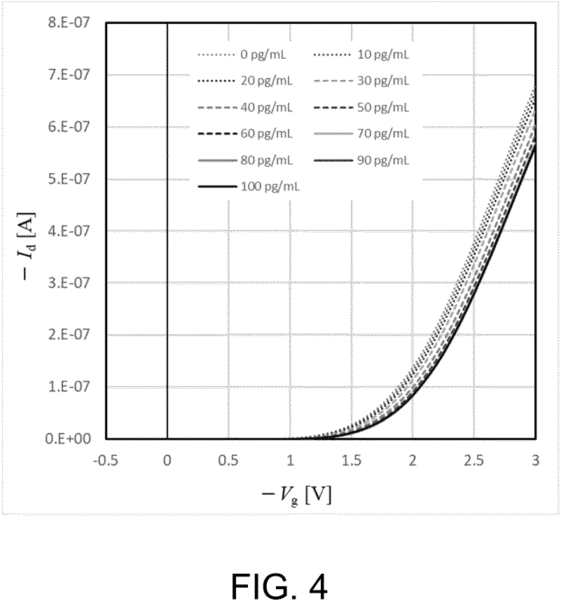

The measurement results are shown in FIG. 4. This is a current-voltage characteristic obtained by averaging three current values measured for each concentration. In FIG. 4, it was found that the current value decreased as the concentration increased.

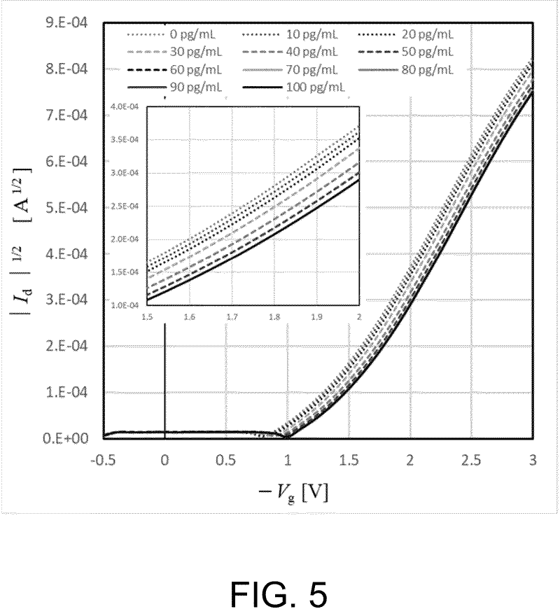

FIG. 5 is a graph in which a vertical axis is the current value to the power of ½, and it is found that the curve shifts along with an increase of the concentration even if a saturation region of a transistor characteristic is expanded.

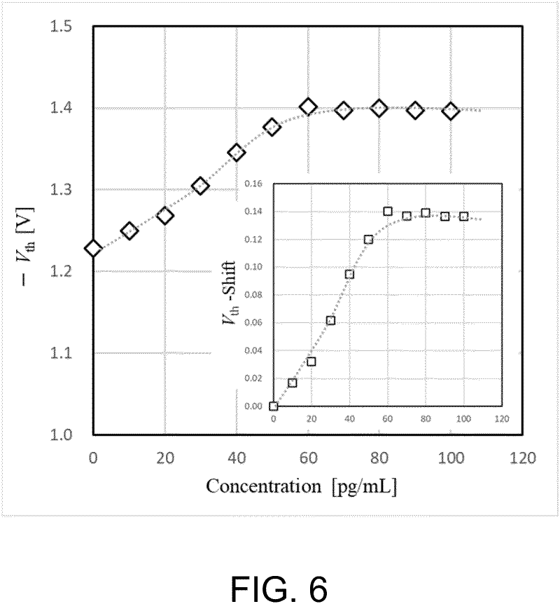

FIG. 6 is a graph in which a vertical axis is the threshold voltage calculated from the current value to the power of ½ and a horizontal axis is the concentration. It was found that the threshold voltage shifted in a negative direction with a change in concentration.

The graph embedded in FIG. 6 is a graph showing the amount of a change in the threshold voltage, in which the threshold voltage Vth(n) at each concentration is normalized with the threshold voltage Vth(0) at a concentration 0. Vth-Shift(n) can be calculated by subtracting Vth(0) from Vth(n) and dividing it by Vth(0). From this graph as well, it was found that the threshold voltage shifted greatly with the change in concentration.

### Oxytocin Detection Experiment: Check of Current Id

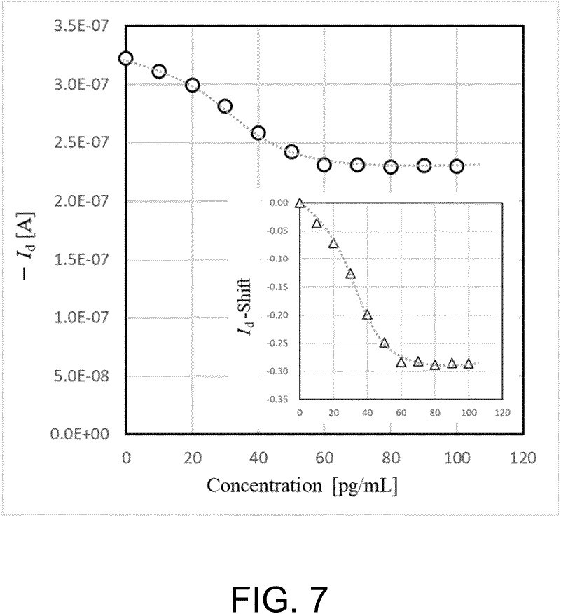

In a detection experiment of Example 1, the obtained current Id is plotted for each oxytocin concentration. The results are shown in FIG. 7. Specifically, FIG. 7 is a graph obtained by averaging three current values measured for each concentration, calculating a voltage corresponding to the current value that was half the current value at -3.0 V at a concentration 0, and obtaining a current value at that voltage for each concentration. It was found that the current value decreased as the concentration increased.

The graph embedded in FIG. 7 is a graph showing the amount of a change in the current value, in which the current Id(n) at each concentration is normalized with the current value Id(0) at a concentration 0. Id-Shift(n) can be calculated by subtracting Id(0) from Id(n) and dividing it by Id(0). From this graph as well, it was found that the current value changed greatly with the change in concentration.

### Comparative Example 1

The detection of the oxytocin was performed using a polymer-immobilized detection electrode that does not have a space shaped like an oxytocin molecule inside.

Specifically, a thin film of gold having a thickness of 100 nm was formed by a vacuum deposition method using a metal mask on a PEN substrate to obtain Electrode 1.

Then, 0.6 mM of dopamine hydrochloride was added to Dulbecco phosphate-buffered saline to prepare Treatment liquid 2. Treatment liquid 2 was put into a container, Electrode 1, a platinum counter electrode, and a Ag/AgCl reference electrode were immersed, and a voltage was applied using cyclic voltammetry. A voltage range was set to -0.5 to 0.5 V, a scan speed was set to 20 mV/s, and this was repeated for 9 cycles. Then, Electrode 1 was washed with ultrapure water and dried to obtain a polymer-immobilized detection electrode.

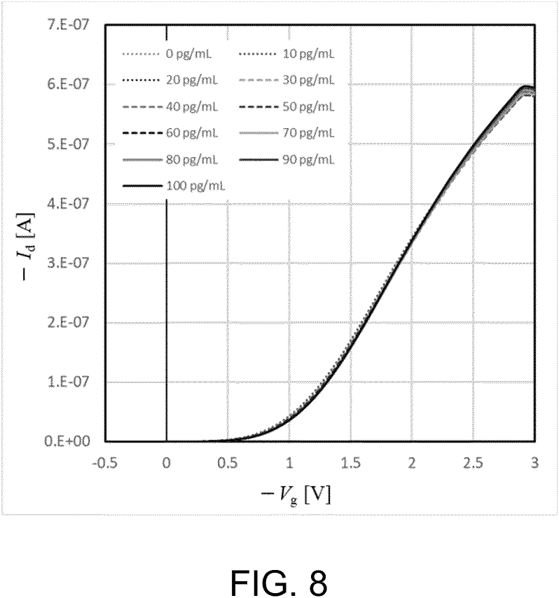

The measurement results (a current-voltage characteristic) are shown in FIG. 8. A relationship that the current value decreased as the concentration increased was not found from FIG. 8.

### Example 2

The same experiment as in Example 1 was performed using a silicon field effect transistor manufactured by ON Semiconductor, manufactured using a normal semiconductor manufacturing process under the product name BSS84, instead of the field effect transistor using the organic semiconductor.

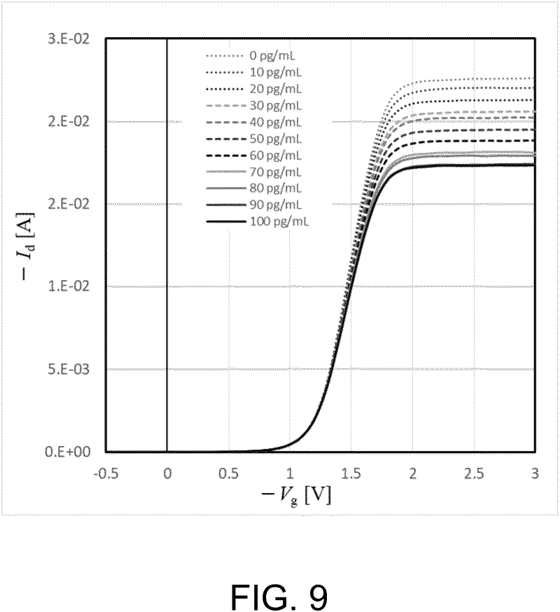

The measurement results (a current-voltage characteristic) are shown in FIG. 9. A relationship that the current value decreased as the concentration increased was found from FIG. 9, and it was shown in FIG. 9 that measurement was possible even in a case where the silicon field effect transistor was used.

### Example 3

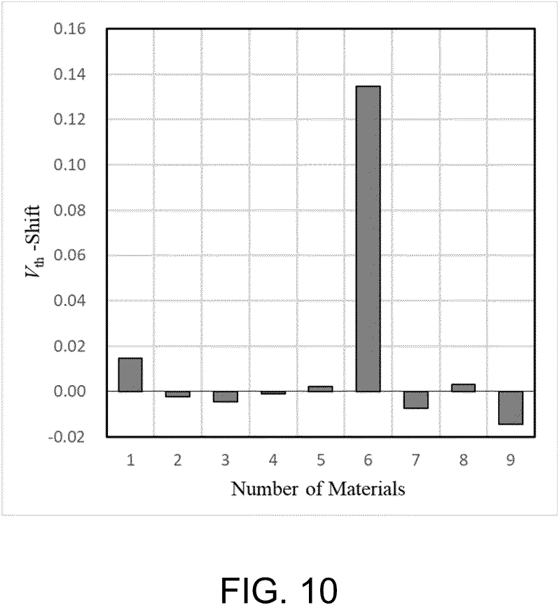

Using the field effect transistor manufactured using the organic semiconductor manufactured in Example 1 and the polymer-immobilized detection electrode having a space shaped like an oxytocin molecule inside, a detection experiment for representative substances contained in saliva was performed on an aqueous solution prepared to have concentrations of the representative substances assumed to be contained in the saliva. Table 1 shows conditions of the substances to be measured, and FIG. 10 shows Vth-Shift(n) calculated from measurement results for each substance. It was shown in FIG. 10 that the polymer-immobilized detection electrode, which has a space shaped like an oxytocin molecule inside, specifically detects oxytocin.

TABLE 1

| No. | Substances | Concentration |
|---|---|---|
| 1 | Albumin | 1.78 g/L |
| 2 | Calcium | 48 mg/L |
| 3 | Creatinine | 0.3 mg/L |
| 4 | D-Glucose | 10 mg/L |
| 5 | L-lactic acid | 18 mg/L |
| 6 | Oxytocin | 100 ng/L |
| 7 | Potassium | 0.55 g/L |
| 8 | Uric acid | 8 mg/L |
| 9 | Vasopressin | 100 ng/L |

The field effect transistor type sensor of the disclosure can quantitatively measure a compound very easily and has industrial applicability.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A transistor type sensor comprising:
a detection electrode that detects a compound by capturing the compound; and
a field effect transistor that has a gate electrode connected to the detection electrode,
wherein a surface of the detection electrode is provided with a film of a molecularly imprinted polymer having a space to which the compound is allowed to bond.

2. The transistor type sensor according to claim 1, wherein each space is formed such that each compound is allowed to be captured in the same orientation.

3. The transistor type sensor according to claim 2, wherein a non-covalently bondable functional group of the molecularly imprinted polymer is exposed in the space toward the captured compound.

4. The transistor type sensor according to claim 1, wherein the molecularly imprinted polymer film is formed of a monomer having an amino group-derived moiety.

5. The transistor type sensor according to claim 1, wherein the compound includes a —SH group, a —S—S— bond, or a —C═C—H group.

6. The transistor type sensor according to claim 5, wherein the compound is oxytocin.

7. The transistor type sensor according to claim 1, wherein the field effect transistor is an organic semiconductor transistor.

8. The transistor type sensor according to claim 1, wherein the field effect transistor is a p-type semiconductor.

9. The transistor type sensor according to claim 1, wherein the transistor type sensor detects the detection target in a solution or dispersion liquid.

10. A manufacturing method for manufacturing the field effect transistor type sensor according to claim 1,
wherein the detection electrode is obtained by a method comprising:
chemically bonding the compound to a surface of a main body of the detection electrode;
applying a monomer-containing solution to the main body of the detection electrode or immersing the main body of the detection electrode in the monomer-containing solution to form the molecularly imprinted polymer;
polymerizing each monomer in the presence of the main body of the detection electrode to form a polymer; and
removing the compound covered with the polymer and chemically bonded to the surface of the main body of the detection electrode to form the molecularly imprinted polymer.

11. The manufacturing method according to claim 10, wherein the polymerization is electrolytic polymerization.

12. The manufacturing method according to claim 10, wherein the removal of the compound covered with the molecularly imprinted polymer and chemically bonded to the surface of the main body of the detection electrode is performed by an electrochemical reaction.

13. A measurement method for measuring a compound to be detected using the field effect transistor type sensor according to claim 1,
wherein the transistor type sensor further comprises a counter electrode, and
wherein the detection electrode and the counter electrode are brought into contact with the detection target.

14. The measurement method according to claim 13, wherein detection is performed in a state where the compound to be detected is included in a solution or a dispersion liquid and the detection electrode and the counter electrode are put into the solution or the dispersion liquid.

15. The measurement method according to claim 13, wherein an applied voltage is a DC voltage.

16. A measurement method for quantitatively measuring a compound using a field effect transistor type sensor, comprising:
bringing a compound to be detected into contact with the detection electrode of the field effect transistor type sensor according to claim 1;
measuring a current Id flowing between a drain electrode and a source electrode of the transistor for each concentration of each compound to obtain a concentration-current Id relationship curve;
bringing the compound having an unknown concentration into contact with the detection electrode to obtain a current Id of the compound having an unknown concentration; and
comparing the current Id of the compound having an unknown concentration with the concentration-current Id relationship curve to determine a concentration for the compound from the current Id having an unknown concentration.

17. A measurement method for quantitatively measuring a compound using a field effect transistor type sensor, comprising:
bringing a compound to be detected into contact with the detection electrode of the field effect transistor type sensor according to claim 1;
measuring a threshold voltage for each concentration of each compound to obtain a concentration-threshold voltage relationship curve;
bringing the compound having an unknown concentration into contact with the detection electrode to obtain a threshold voltage of the compound having an unknown concentration; and
comparing the threshold voltage of the compound having an unknown concentration with the concentration-threshold voltage relationship curve to determine a concentration for the compound from the threshold voltage having an unknown concentration.

18. The measurement method according to claim 17,
wherein the measuring of the threshold voltage includes
applying a voltage Vd to a drain electrode with a source electrode of the field effect transistor as a reference and applying a voltage Vg to a counter electrode with the source electrode as a reference;
sweeping the Vg and measuring a current Id flowing between the drain electrode and the source electrode to obtain a Vg-Id curve; and
using the Vg-Id curve to obtain a value of the threshold voltage.

19. The measurement method according to claim 16,
wherein the transistor type sensor further comprises a counter electrode, and
wherein the detection electrode and the counter electrode are brought into contact with the compound.

20. The measurement method according to claim 16, wherein detection is performed in a state where the compound is included in a solution or a dispersion liquid and the detection electrode and the counter electrode are put into the solution or the dispersion liquid.

21. The measurement method according to claim 16, wherein an applied voltage is a DC voltage.
